(19) United States
(12) Patent Application Publication
(10) Pub. No.: US 2003/0027745 A1
(43) Pub. Date: Feb. 6, 2003

(54) Diagnostic and prognostic method for evaluating ocular inflammation and oxidative stress and the treatment of the same

(76) Inventor: John E. Repine, Englewood, CO (US)

Correspondence Address: SHERIDAN ROSS PC, 1560 BROADWAY, SUITE 1200, DENVER, CO, 80202

(21) Appl. No.: 10/172,364

(22) Filed: Jun. 13, 2002

Related U.S. Application Data
(60) Provisional application No. 60/298,231, filed on Jun. 13, 2001.

(51) Int. Cl. C12Q 1/26; A61K 31/00

(52) U.S. Cl. 514/1; 435/25

(57) ABSTRACT

Oxidative stress and inflammation often occur in the eye and often in association with aging or ocular disease. Increases in blood oxidative stress and inflammatory factors can be used to identify individuals with increased ocular oxidative stress and inflammation, select and guide appropriate interventions or treatments, identify selected patients for clinical study and/or determine the efficacy of various interventions or treatments. The present invention describes such factors and will improve the diagnosis, treatment and prevention of ocular oxidative stress and/or inflammation and related ocular conditions.

### PRIORITY CLAIM

This application claims priority from U.S. Provisional Patent Application No. 60/298,231, filed Jun. 13, 2001, entitled “A Diagnostic and Prognostic Method for Evaluating Ocular Inflammation and Oxidative Stress and the Treatment of the Same”. This application also claims priority from U.S. Provisional Patent Application No. 60/147,965 filed on Aug. 9, 1999 and U.S. patent application Ser. No. 09/635,517, filed Aug. 9, 2000, both entitled “Method for the Treatment of Ocular Oxidative Stress”. The entire disclosure of the priority applications are considered to be part of the disclosure of the accompanying application and are hereby incorporated by reference.

### FIELD OF THE INVENTION

This invention relates to a method for assessing, predicting and preventing ocular inflammation and oxidative stress in the eye. More particularly, this invention relates to using repeatable blood and other analyses to evaluate the status and the nature of inflammation and oxidative stress that occurs in the eye in good health as well as during aging and disease.

### BACKGROUND OF THE INVENTION

The eye is a complex, highly vascular, heavily enervated, and environmentally exposed organ that is frequently subjected to numerous events that produce inflammation and/or oxidative stress during various stages of a mammal's health, disease, and aging. Inflammation and/or oxidative stress can rapidly alter or damage key cellular components that are in the eye, including lipids, proteins, RNA and DNA. In addition, inflammation and/or oxidative stress can impair the function of various parts of the eye, such as the lens, retina, iris, vitreous, blood vessels and nerves. Inflammation and/or oxidative stress can produce eye dysfunction and damage by many processes including proliferation and injury to blood vessels, cellular apoptosis or necrosis, increased ocular pressures, lipid peroxidation, lens opacification. Additionally, biochemical reactions involving cycloxygenase, nitric oxide (NO) synthase and other enzymatic reactions can lead to oxidative stress. Enhanced mitochondrial metabolism of oxygen also produces superoxide anion (O₂⁻) and other reactive species of oxygen. Exogenous sources of oxidative stress include cigarette smoking, various drugs and photooxidative reactions caused by irradiation to the eye as a consequence of sunlight or laser therapy. Moreover, even without an increased production or exposure to oxidants, the eye might be exposed to increased oxidative stress if any of a number of antioxidants or factors that reduce the toxicity of oxidative stress are decreased or impaired. For example, aging has been reported to decrease levels of the antioxidants and anti-inflammatory agents, such as glutathione (GSH) and taurine, respectively. The accumulation of iron (ferritin) that occurs with aging is also believed to increase oxidative stress since uncomplexed iron facilitates the reaction of superoxide (O₂⁻) and hydrogen peroxide (H₂O₂) and produces the highly toxic hydroxyl radical (OH). Furthermore, oxidative stress, cigarette smoke, and other factors mobilize Fe⁺⁺ from ferritin and other complexing proteins.

Oxidative stress occurs in the eye during both health and disease. For example, during normal circumstances, the mitochondrial consumption of O₂ generates oxygen radicals which may not be completely detoxified by antioxidants and consequently may produce oxidative stress. Another example is apparent in age-related macular degeneration or ARMD, the leading cause of blindness in the elderly, a condition in which deposits of an oxidized lipid, lipofuscin, are manifest in the retina. Levels of hydrogen peroxide (H₂O₂) are also increased in the vitreous of the eye and are believed to contribute to cataract, glaucoma and/or other ocular abnormalities. Following surgery, laser therapy, infection, and/or injury, the eye is often subjected to reactions that generate inflammation activating immune and inflammatory cells and other cellular functions that produce oxidative stress. Fortunately, treatment and prevention strategies are available and are being developed that have the potential of reducing the undesirable effects of inflammation and/or oxidative stress on the eye. However, at present, it is very difficult and largely unascertainable to determine what type, how much and/or when inflammation and oxidative stress is occurring in the eye, and consequently when, which and/or how much intervention is needed to optimally modify these unwanted effects. Accordingly, approaches that enable assessment of ocular inflammation and/or oxidative stress would be valuable with respect to providing and improving (a) identification and quantification of ocular oxidative stress and/or inflammation, (b) selection of better defined patients who may be more similar in their disease symptoms, activity and/or progression and thus much better characterizable for clinical study, (c) prediction of the development of ocular oxidative stress and/or inflammation in ways that will enable more appropriate, earlier and/or more prophylactic use of interventions that modulate these processes and their consequences, and (d) assessment of the responses to various interventions with respect to their effects on inflammation, oxidative stress and disease progression or severity.

There are many possible sources and generators of inflammation and/or oxidative stress in the eye. The list encompasses phagocytic cells (e.g. neutrophils, macrophages, monocytes, lymphocytes, and eosinophils), enzyme systems (e.g. xanthine oxidase) and non-enzymatic chemical reactions (e.g. drugs). Mitochondrial respiration can also contribute to oxidative stress. Cytokines (e.g. interleukins and growth factors, proteases) and reactive oxygen species (e.g. superoxide anion, hydroxyl radical, and nitrogen based intermediates) are additional examples of possible contributors to inflammation and oxidative stress in the eye. These contributors and other measurable factors could be produced by cellular and other mechanisms within the eye (e.g. retinal endothelial cells), cells (e.g. phagocytes) and/or other factors which transit from the blood into the eye. These events can cause or result from other changes that are occurring in the eye including alterations in blood flow (hypoxia-reoxygenation) and nutrients.

Endogenous and exogenous insults can initiate the processes that lead to ocular inflammation and/or oxidative stress. Some examples include aging, disease, cigarette smoke, irradiation, surgery, laser therapy, trauma, infection, foreign body exposure, inhalation of high concentrations of oxygen, contact with toxins, pharmacologics and/or nutraceutical agents.

Inflammation and oxidative stress often develop in the eye in association with acquired ocular disorders, such as infection, cataract development, age-related macular degeneration (ARMD), diabetic retinopathy, other retinopathies, iritis, uveitis, keratitis, vasculitis, glaucoma, other acquired ocular abnormalities, and congenital or genetic disorders such as Stargardt's Macular Dystrophy. By way of example, levels of hydrogen peroxide (H₂O₂) have been measured in the vitreous fluid of the eye and associated with cataract. However, the presence of H₂O₂ in the vitreous is usually assessed only by an invasive technique which requires sampling the fluid of the open eye when a cataract is being removed surgically. This approach is not valuable since it is invasive, not repeatable and obviously unable to predict cataract development at a time when preventive measures could be instituted. In addition, the effect of aging, disease or interventions can not be easily evaluated at different points in time if the only method of evaluation is an invasive approach that requires eye surgery. However, if evaluation methods were available to non-invasively assess ocular H₂O₂ levels, it is possible that treatment could be initiated and monitored for its effect on H₂O₂, and H₂O₂ related processes in individuals before an ocular disease has an opportunity to progress. In addition, if evaluation methods were available to non-invasively assess ocular H₂O₂ levels, the methods could be repeated to provide quantitative measurements showing whether the individual is responding to treatment. Conversely, individuals with normal H₂O₂ levels may not need intervention since their risk of developing H₂O₂, dependent events, such as cataract, might reasonably be reduced and treatments directed at decreasing H₂O₂ levels per se might not be effective. Since all treatments involve expense and risk, it is not practical or appropriate to treat individuals for conditions which are not present.

In summary, inflammation and/or oxidative stress frequently occurs in the eye and often in association with detrimental effects. These adverse effects can decrease vision by damaging key eye structures by processes that may involve any of a number of different processes. Treatments and preventive strategies are available and being developed which have the potential of limiting inflammation and/or oxidative stress and the related events that impair the eye and vision. Thus, as will be described herein, a non-invasive, repeatable way of assessing the status and nature of oxidative stress and inflammation and related diseases and their response to interventions is needed.

The eye has generally been considered to be relatively isolated from the rest of the body and many believe that events that occur in the eye would not be reflected in the blood. In addition, it is commonly believed that systemic processes may not contribute to ocular disorders per se even though some of these system events damage other organs. There are no known ways of predicting ocular oxidative stress and/or inflammation by analyses of blood samples. Consequently, the present invention runs contrary to the present beliefs regarding the interconnectedness between the eye and the blood as well as present knowledge about the isolated nature of events related to inflammation and oxidative stress in the eye.

### DISCLOSURE OF THE INVENTION

The present invention provides a method of measuring, predicting, diagnosing, assessing the progression of, assessing the treatment of, and preventing ocular oxidative stress and inflammation by assessing a value of at least one indicator factor and either assessing a second value and comparing the second to the first or comparing the value to baseline values and treating the ocular oxidative stress and inflammation, if present. The present invention also provides a method of measuring, predicting, diagnosing, assessing the progression of, assessing the treatment of, and preventing ocular disease by assessing a value of at least one indicator factor and either assessing a second value and comparing the second to the first or comparing the value to baseline values and treating the ocular disease, if present. The present invention further presents a method of monitoring and recognizing various indicator factors which can lead to or assist in evaluating oxidative stresses and/or ocular inflammation. The present invention also involves the identification and monitoring of various blood factors which can be used to indicate that oxidative stresses are occurring in a mammal. Certain blood factors, including but not limited to, reduced glutathione (“GSH”) levels, oxidized glutathione (“GSSG”), nitric oxide (“NO”) levels, vascular endothelial growth factor (“VEGF”) levels, and nuclear factor kappa-b (“NFκB”) activity, may present methods of identifying, monitoring and evaluating oxidative stresses and ocular inflammation. These factors can be analyzed alone and/or in combination with other factors. These factors can also be analyzed as ratios; for example, the ratio of GSSG to GSH can be analyzed.

Studies were performed utilizing patients with Age-Related Macular Degeneration (“ARMD”). ARMD occurs in two forms: dry or wet. In dry ARMD, the retinal cells in or near the macula appear to be damaged. As these cells are perturbed, the central vision may become affected. In wet ARMD, blood vessels behind the retina grow toward the macula and disturb vision. Because these new blood vessels are fragile, they will often break and leak blood and fluid under the macula. This causes rapid damage to the macula and can lead to a loss of central vision in a short period of time. ARMD is the leading cause of blindness in the elderly and is believed to involve increased ocular oxidative stress and inflammation. There is evidence of increased lipofuscin, an oxidized lipid, in the retina in patients with ARMD. In addition, ARMD is also associated with aging and cigarette smoking, two factors which are associated with increased inflammation and oxidative stress. Thus, ARMD patients present a good model of ocular oxidative stress and inflammation for study. The performed studies indicate that the above-listed factors, as well as others, may present eye professionals with methods to identify, evaluate and monitor oxidative stresses and/or ocular inflammation in patients, even before symptoms of such stresses and/or inflammation present themselves.

The invention further discloses methods of using blood and other analyses to assess the effects of diet, pharmacologic, nutraceutical, laser and/or other interventions and treatments that affect ocular oxidative stress and/or inflammation directly or indirectly. All of the genes, proteins, lipids, substances or elements that constitute or regulate these and related factors are candidates for measurement by various techniques for the purposes of identification, measurement or assessment of ocular inflammation or oxidative stresses.

### OBJECTS OF THE INVENTION

It is thus an object of this invention to provide a non-invasive method of monitoring factors which can cause oxidative stress and/or ocular inflammation.

It is also an object of this invention to provide a non-invasive method of assessing the responsiveness of individuals to certain treatments typically used to treat diseases resulting from ocular inflammation and/or oxidative stresses.

Additionally, it is an object of this invention to provide a non-invasive method of measuring various blood factors which can be indicative of oxidative stress, with the method being easily replicated.

It is further an object of the present invention to provide a non-invasive method of predicting ocular inflammation or oxidative stresses based on blood factors and other analyses to prevent symptoms of ocular inflammation or oxidative stress appear.

It is a still further object of the present invention to provide a set of factors which can be identified, monitored and assessed repeatedly and are correlated to oxidative stresses in the eye and/or ocular inflammation.

These and other objects, features, and advantages of the invention will become apparent from the following best mode description, the drawings and the claims.

### BRIEF DESCRIPTION OF THE DRAWINGS

The figures which follow depict a preferred embodiment of the invention, and may depict various alternative embodiments. The invention is not limited to the embodiment or embodiments depicted herein since even further various alternative embodiments will be readily apparent to those skilled in the art.

FIG. 1 is a chart showing the decreased reduced blood glutathione (GSH) levels in an Age-Related Macular Degeneration patient, prior to and following treatment of the patient with N-acetylcysteine (NAC).

FIG. 2 is a photographic view and chart showing the decreased number of drusens, a retinal abnormality, in an ARMD patient following NAC treatment.

FIG. 3 is a chart showing the increased risk of ARMD in women 75 years of age and above.

FIG. 4 is a chart showing that the NFκB activity levels are increased in ARMD patients versus control subjects.

FIG. 5 is a chart showing that the blood nitric oxide levels are increased in ARMD patients versus control subjects.

FIG. 6 is a chart showing that VEGF levels are increased in ARMD patients versus control subjects.

### BEST MODE FOR CARRYING OUT THE INVENTION

At the outset, it should be understood that this invention comprises a method of assessing, evaluating, monitoring, predicting, and/or preventing oxidative stresses and/or ocular inflammation in the eye utilizing non-invasive and repeatable blood and other analyses. The description which follows describes a preferred embodiment of the invention, and various alternative embodiments. It should be readily apparent to those skilled in the art, however, that various other alternative embodiments may be accomplished without departing from the spirit or scope of the invention. The following description is provided to enable any person who is skilled in the art to which the present invention pertains, or with which it is most nearly connected, to make and use the same, and sets forth in specific and conceptual terms, the best mode contemplated now by the inventors for the purpose of carrying out their invention.

It is to be noted that the term “a” or “an” entity refers to one or more of that entity; for example, a protein refers to one or more proteins, or to at least one protein. As such the terms “a” (Or “an”), “one or more” and “at least one” can be used interchangeably herein. It is also to be noted that, for the purposes of this discussion, the terms “comprising”, “including”, and “having” can be used interchangeably.

The eye is a very inaccessible organ to study. As a result, it is very difficult to quantify inflammation and/or oxidative stress in the eye, to measure changes in inflammation or oxidative stress, and to determine how changes in these parameters reflect and contribute to aging and disease. It is also difficult to determine the effect of various interventions on inflammation and/or oxidative stress in the eye.

Oxidative stress is defined as an increase in the production of highly reactive species of oxygen and/or nitrogen (“free radicals”) and/or a decrease in antioxidant defense systems. Antioxidant defense systems encompass enzymatic and non-enzymatic detoxifying mechanisms, binders of various cofactors, such as metals that facilitate oxidative reactions, and molecular alterations that make cellular lipids, membranes or DNA less susceptible to damage by oxidants.

To support the findings of this invention, several examples of studies shall be presented. For each of the studies, patients having ARMD are compared with control subjects. Numerous factors have been identified, through these studies, as being correlated to oxidative stress and/or ocular inflammation. However, it should be noted that other factors that can be analyzed in blood or in cellular function may be shown to be correlated to eye functions and that the measurement and monitoring of such factors are intended to be within the scope of this invention.

The present invention teaches that patients with ARMD possess decreased levels of glutathione (GSH). It is known that GSH levels decrease in cells that are exposed to oxidative stress and inflammation. Decreases in GSH lead to even further increased oxidative stresses. However, the ability of alterations in GSH, GSSG (the oxidized form of GSH) or their ratios to predict or reflect ARMD development, progression or response to treatment has never been advanced or demonstrated. Since it shown that patients with ARMD, as opposed to the control subjects, have decreased levels of GSH and that the levels of GSH increased following NAC treatment, alterations in GSH, GSSG and other factors when analyzed alone or in combination, can be used to help identify ARMD and other patients who have increased ocular oxidative stress and/or inflammation. For example, these responses may occur in the eye of ARMD or other patients as the disease progresses from its dry to wet forms or from less serious to more serious wet forms. Consequently, measurements of these factors could provide a way of diagnosing and following the progression of ARMD or other conditions in patients, especially when the measurements are made sequentially and compared repeatedly in the same individual.

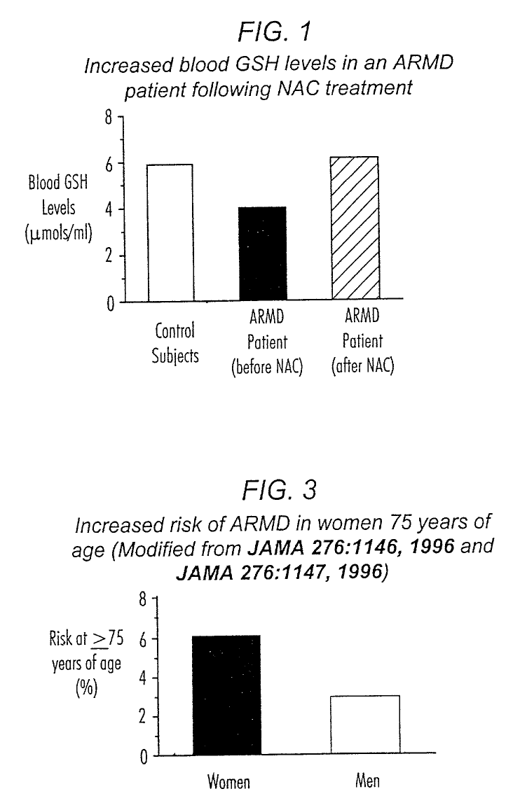

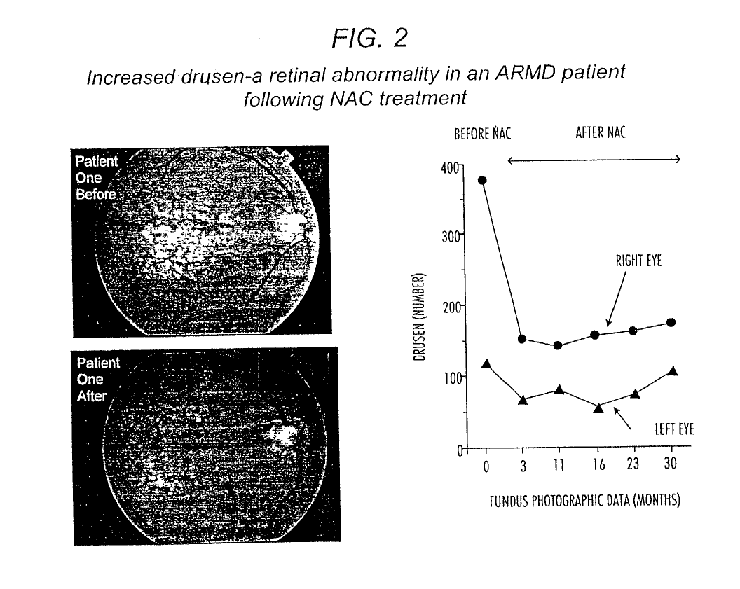

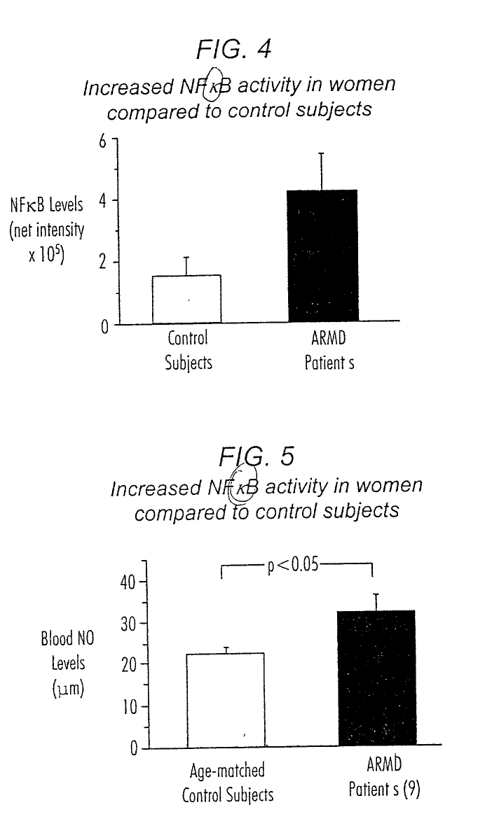

A second aspect of the invention is that the effectiveness of treatments that alter oxidative stress can be assessed by the measurement, especially sequential measurement, of blood factors. In addition, we found that an ARMD patient taking N-acetylcysteine (NAC) had increased GSH levels compared to the decreased GSH levels of the ARMD patient before taking NAC (FIG. 1). NAC is a nutraceutical agent which increases GSH levels and increased GSH levels decrease oxidative stress. Thus, measurement of the appropriate blood factors can also reflect a successful response to treatment (responders). This is evidenced by the decreased drusen (believed to be at least in part an oxidized lipid product) observed in an ARMD patient following NAC treatment (FIG. 2). Drusen are retinal abnormalities that are believed to be hyaline nodules or colloid bodies deposited in Bruch's membrane that separates the inner choroidal vessels from the retinal pigment epithelium. Drusen range from small and discrete to large irregularly shaped bodies with indistinct edges. Drusen often occur in patients with ARMD.

Alternatively, the failure of GSH levels to increase in response to NAC treatment identifies individuals who are not affected by NAC treatment for whatever reasons and individuals whose oxidative stress levels may not have been decreased by the NAC treatment (non-responders). In this way, measurement of these levels can be used not only to predict events occurring in patients undergoing oxidative stress but also to reflect a response or non-response to a therapeutic or other intervention. Consequently, the measuring and quantification of GSH levels in patients enables identification of subpopulation of individuals who may be advancing more rapidly in their disease and/or who may be more responsive to one or another therapy. Use of baseline measurements in combination with other factors could have additional power in making and refining these predictions and reflections. The specific factor or combination of factors that are measured for this purpose will be selected depending on the specific condition and/or the specific therapy(s) that is (are) being evaluated and the particular disease or disorder being evaluated. As described, the approach can provide an individualized sequential method for evaluating patients before and during treatment and comparing their responses to other patients being treated with the same or other interventions. The approach enables one to decide if a particular therapy is useful in a specific individual and to select an appropriate therapy for each individual based on a quantifiable and repeatable biochemical response.

Another factor identified in the studies as being correlated to oxidative stress and ocular inflammation is nuclear factor kappa-b (“NFκB”). Nuclear factor-κB (NFκB) is a heterodimeric transcription factor complex that mediates multiple immune and inflammatory responses. The activated form of NFκB consists of two proteins, a p65 (aka rel A) subunit and a p50 subunit. Rel, rel B, c-rel and p52 may also be a part of NFκB and different forms of activated NFκB may activate different sets of target genes. Activation of NFκB in the cytoplasm is associated with a series of events that facilitate the transport of NFκB to the nucleus when it has its effects on centrally controlled genetic processes.

Many stimuli, including cytokines, protein kinase C activators, viruses and oxidants, activate NFκB (see Table I).

TABLE I
Stimuli That Activate NFκB

Cytokines

Tumor necrosis factor α
Interleukin-1β
Interleukin-17

Protein kinase C activators

Phorbol esters
Platelet-activating factor

Oxidants

Hydrogen peroxide
Ozone

Viruses

Rhinovirus
Influenzavirus
Epstein-Barr virus
Cytomegalovirus
Adenovirus

Immune stimuli

Phytohemagglutinin
Anti-CD3 antibodies (by means of T-lymphocyte activation)
Antigen

Other

Lipopolysaccharide
Ultraviolet radiation

Agents that inhibit NFκB activation include pyridoxine dithiocarbamate, NAC, glucocorticoids, acetosalycyclic acid, sodium salicylate, glitoxin, interleukin-10 and gold salts. Additional inhibitors of NFκB include inhibitors of IκB kinases or certain factors, such as NEMO, that alter kinase related and other factors involved in NFκB activation. For example, IκBα or IκBβ superrepressor, especially non-phosphatable forms offer a way of inhibiting NFκB. Compounds (e.g. peptides) or strategies that block nuclear localization and/or nuclear uptake of NFκB can be used to effectively inhibit NFκB activity. NFκB can also be inhibited by factors such as P300 creb binding protein that prevent NFκB from interacting with any basic transcription complex.

As shown in Table II, NFκB regulates expression of many genes, oftentimes in conjunction with the nuclear factor of interleukin-6 (NF-IL-6) and activator protein (AP-1). NFκB also acts on genes for granulocyte colony stimulator factor (GCSF), various chemokines (e.g., IL-8), adhesion molecules (e.g. E. Selectin, ICAM), growth factors (e.g. VEGF), inflammation inducing factors (e.g. tumor necrosis factor (TNF), IL-1 IL-6) and enzymes (e.g. nitric oxide (NO) synthase and cyclooxygenase).

TABLE II
Some Proteins Regulated by NFκB

Proinflammatory cytokines

Tumor necrosis factor α
Interleukin-1β
Interleukin-2
Interleukin-6
Granulocyte-macrophage colony-stimulating factor
Macrophage colony-stimulating factor
Granulocyte colony-stimulating factor

Chemokines

Interleukin-8
Macrophage inflammatory protein 1α
Macrophage chemotatic protein┐
Gro-α, -β, and -γ
Eotaxin

Inflammatory enzyes

Inducible nitric oxide synthase
Inducible cyclooxygenase-2
5-Lipoxygenase
Cytosolic phospholipase A₂

Adhesion molecules

Intercellular adhesion molecule┐
Vascular-cell adhesion molecule┐
E-selectin

Receptors

Interleukin-2 receptor (α chain)
T-cell receptor (β chain)

The comprehensive effects of NFκB leads to a multidimensional array of events involving many factors which can be modulated at a central level. By way of example, one complex scheme could involve elaboration of neutrophils from the bone marrow, production of chemotaxins, recruitment and activation of neutrophils and the release of enzymes and oxidants from neutrophils. The latter is facilitated by COX2 (prostaglandins, thromboxanes), NO synthase and other molecules which increase blood flow and blood vessel permeability. Thus, the present invention is partially directed to the ability to modulate the effects of the central coordinating effector NFκB which, in turn, will significantly reduce all of these events and the many untoward effects including apoptosis, necrosis, and cell differentiation that are unwanted consequences of oxidative stress.

For unknown reasons, the risk of ARMD is greater in women over 75 years of age than in men over 75 years of age (FIG. 3). Thus, the levels of NFκB which control and reflect many inflammatory and oxidative stress related processes were increased more in women than in men.

This association suggests a relationship between blood NFκB levels and the incidence/severity of oxidative stress and inflammation in the eye and ARMD especially since oxidative stress and inflammation could contribute to ARMD. This finding suggests that certain women may be more responsive to approaches that decrease NFκB than other women or men. Because NFκB controls many inflammatory and oxidative stress responses, changes in NFκB or other factors in the blood of individuals with or without the prospect of developing cataracts, diabetic retinopathy, glaucoma or other ocular disorders including aging, or other conditions as related to oxidative stress and inflammation may be similar and reflected similarly by blood or other assessments.

The presumed inflammatory and oxidative stress nature of most ocular disorders indicates that measurement of any or all these factors related to these processes alone or in combination may be useful as methods of assessing predicting and/or reflecting changes that occur in individuals. Additionally, the present invention identifies other factors that may accomplish the stated objectives.

For instance, blood nitric oxide (NO) levels were increased in patients with ARMD compared to age-matched control subjects (FIG. 5). NO is a factor that could affect blood vessel integrity, inflammation, blood flow and/or oxidative stress. Reaction of NO with superoxide anion (O₂⁻) produces peroxinitrite (OONO) which is a powerful agonist for many different mechanisms that are likely to contribute to ocular oxidative stress and/or inflammation. NO is also involved in the production of a number of cytokines, inflammatory growth and other phlogistic factors that impart the eye. Evaluation of NO could improve assessment, prediction and/or responses to various interventions in individuals susceptible to ocular oxidative stress and its consequences. NO is a prominent component of cigarette smoke and cigarette smoking is associated with ARMD development.

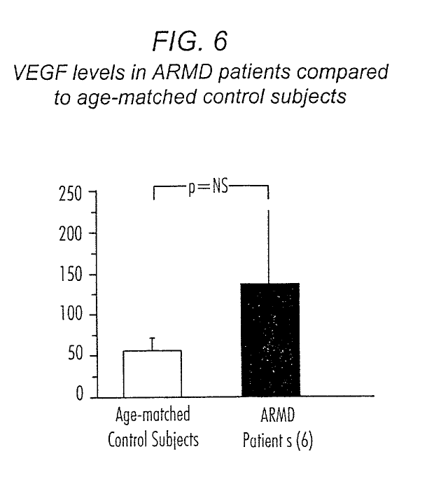

Further, the present invention teaches that increased VEGF levels in patients with ARMD exist compared to control subjects (FIG. 6). Although the groups are not different statistically, the large individual variation in the values for the ARMD patients points to the possibility that these individual assessments may predict individuals who are at greater or lesser risk to the development of ARMD, ARMD complications or other ocular disorders. The wet phase of ARMD is characterized in part by the proliferation of blood vessels in the retina and VEGF is a factor which increases vascular proliferations. Thus, elevations in blood VEGF levels might reflect elevated VEGF and related events in the eye.

The methods for measuring oxidative stress and inflammation, with specific reference to the above-identified factors, include all techniques (e.g. biochemical, molecular, cellular and physiologic) typically used for assaying genes (DNA, RNA), proteins, lipids, blood or other substances. In some cases, the approach may depend on separating particular cells or factors from the blood and then analyzing them in vitro and in vivo test systems to assess the effect.. Statistical analyses will be conducted to determine significant associations and identify combinations of factors that have greater usefulness compared to single factors. Ratios of factors, for example, arginase to NO, may be useful in some cases. While various embodiments of the present invention have been described in detail, it is apparent that further modifications and adaptations of the invention will occur to those skilled in the art. However, it is to be expressly understood that such modifications and adaptations are within the spirit and scope of the present invention.

We claim:

1. A method for measuring ocular oxidative stress and inflammation in mammals comprising the steps of:
assessing a first value of at least one indicator factor in said mammals wherein said at least one indicator factor is correlated to ocular oxidative stress and inflammation;
assessing a second value of the at least one indicator; and,
comparing said second value to said first value of the at least one indicator factor.

2. A method for measuring ocular oxidative stress and inflammation in mammals as described in claim 1 wherein the at least one indicator factor is a level of reduced glutathione (GSH) in said mammal's blood.

3. A method for measuring ocular oxidative stress and inflammation in mammals as described in claim 1 wherein the at least one indicator factor is a level of nuclear factor kappa-b (NFκB) activity in said mammal.

4. A method for measuring ocular oxidative stress and inflammation in mammals as described in claim 1 wherein the at least one indicator factor is a level of nitric oxide (NO) in said mammal's blood.

5. A method for measuring ocular oxidative stress and inflammation in mammals as described in claim 1 wherein the at least one indicator factor is a level of vascular endothelial growth factor (VEGF) in said mammal's blood.

6. A method for measuring ocular oxidative stress and inflammation in mammals as described in claim 1 wherein the at least one indicator factor is a level of oxidized glutathione (GSSG) in said mammal's blood.

7. A method for measuring ocular oxidative stress and inflammation in mammals as described in claim 1 wherein the at least one indicator factor is a ratio of a level of oxidized glutathione (GSSG) to a level of reduced glutathione (GSH) in said mammal's blood.

8. A method for measuring ocular oxidative stress and inflammation in mammals as described in claim 1 wherein said at least one indicator factor is selected from a group consisting of: cytokines, genes, proteins, lipids, minerals, growth factors, proteases, oxidants, antioxidants, phlogistins, metals, or inhibitors.

9. A method for assessing the effectiveness of treatment of ocular oxidative stress and inflammation comprising the steps of:
assessing a first value of at least one indicator factor in said mammals wherein said at least one indicator factor is correlated to a treatment of ocular oxidative stress and inflammation;
treating said oxidative stress and inflammation;
assessing a second value of said at least one indicator factor; and,
comparing said second value to said first value of the at least one indicator factor.

10. A method for assessing the effectiveness of treatment of ocular oxidative stress and inflammation as described in claim 9 wherein the at least one indicator factor is a level of reduced glutathione (GSH) in said mammal's blood.

11. A method for assessing the effectiveness of treatment of ocular oxidative stress and inflammation as described in claim 9 wherein the at least one indicator factor is a level of nuclear factor kappa-b (NFκB) activity in said mammal.

12. A method for assessing the effectiveness of treatment of ocular oxidative stress and inflammation as described in claim 9 wherein the at least one indicator factor is a level of nitric oxide (NO) in said mammal's blood.

13. A method for assessing the effectiveness of treatment of ocular oxidative stress and inflammation as described in claim 9 wherein the at least one indicator factor is a level of vascular endothelial growth factor (VEGF) in said mammal's blood.

14. A method for assessing the effectiveness of treatment of ocular oxidative stress and inflammation as described in claim 9 wherein the at least one indicator factor is a level of oxidized glutathione (GSSG) in said mammal's blood.

15. A method for assessing the effectiveness of treatment of ocular oxidative stress and inflammation as described in claim 9 wherein the at least one indicator factor is a ratio of a level of oxidized glutathione (GSSG) to a level of reduced glutathione (GSH) in said mammal's blood.

16. A method for assessing the effectiveness of treatment of ocular oxidative stress and inflammation as described in claim 9 wherein the at least one indicator factor is selected from a group consisting of: cytokines, genes, proteins, lipids, minerals, growth factors, proteases, oxidants, antioxidants, phlogistins, metals, or inhibitors.

17. A method for assessing the effectiveness of treatment of ocular oxidative stress and inflammation as described in claim 9 wherein the treatment comprises administration of N-acetylcysteine to said mammal.

18. A method of predicting ocular oxidative stress and inflammation in mammals comprising the steps of:
assessing a value of at least one indicator factor in said mammals wherein said at least one indicator factor is correlated to a treatment of ocular oxidative stress and inflammation; and,
comparing said first value of the at least one indicator factor to baseline values in other mammals in various stages of ocular oxidative stress and inflammation.

19. A method of predicting ocular oxidative stress and inflammation in mammals as described in claim 18 wherein the at least one indicator factor is a level of reduced glutathione (GSH) in said mammal's blood.

20. A method of predicting ocular oxidative stress and inflammation in mammals as described in claim 18 wherein the at least one indicator factor is a level of nuclear factor kappa-b (NFκB) activity in said mammal.

21. A method of predicting ocular oxidative stress and inflammation in mammals as described in claim 18 wherein the at least one indicator factor is a level of oxidized glutathione (GSSG) in said mammal's blood.

22. A method of predicting ocular oxidative stress and inflammation in mammals as described in claim 18 wherein the at least one indicator factor is a level of vascular endothelial growth factor (VEGF) in said mammal's blood.

23. A method of predicting ocular oxidative stress and inflammation in mammals as described in claim 18 wherein the at least one indicator factor is a level of nitric oxide (NO) in said mammal's blood.

24. A method for predicting ocular oxidative stress and inflammation in mammals as described in claim 18 wherein the at least one indicator factor is a ratio of a level of oxidized glutathione (GSSG) to a level of reduced glutathione (GSH) in said mammal's blood.

25. A method of predicting ocular oxidative stress and inflammation in mammals as described in claim 18 wherein the at least one indicator factor is selected from a group consisting of: cytokines, genes, proteins, lipids, minerals, growth factors, proteases, oxidants, antioxidants, phlogistins, metals, or inhibitors.

26. A method of diagnosing ocular oxidative stress and inflammation in mammals comprising the steps of:
assessing a value of at least one indicator factor in said mammals wherein said at least one indicator factor is correlated to a treatment of ocular oxidative stress and inflammation; and,
comparing said value of the at least one indicator factor to baseline values in other mammals with ocular oxidative stress and inflammation.

27. A method of diagnosing ocular oxidative stress and inflammation in mammals as described in claim 26 wherein the at least one indicator factor is a level of reduced glutathione (GSH) in said mammal's blood.

28. A method of diagnosing ocular oxidative stress and inflammation in mammals as described in claim 26 wherein the at least one indicator factor is a level of nuclear factor kappa-b (NFκB) activity in said mammal.

29. A method of diagnosing ocular oxidative stress and inflammation in mammals as described in claim 26 wherein the at least one indicator factor is a level of nitric oxide (NO) in said mammal's blood.

30. A method of diagnosing ocular oxidative stress and inflammation in mammals as described in claim 26 wherein the at least one indicator factor is a level of oxidized glutathione (GSSG) in said mammal's blood.

31. A method of diagnosing ocular oxidative stress and inflammation in mammals as described in claim 26 wherein the at least one indicator factor is a level of vascular endothelial growth factor (VEGF) in said mammal's blood.

32. A method of diagnosing ocular oxidative stress and inflammation in mammals as described in claim 26 wherein the at least one indicator factor is a ratio of a level of oxidized glutathione (GSSG) to a level of reduced glutathione (GSH) in said mammal's blood.

33. A method of diagnosing ocular oxidative stress and inflammation in mammals as described in claim 26 wherein the at least one indicator factor is selected from a group consisting of: cytokines, genes, proteins, lipids, minerals, growth factors, proteases, oxidants, antioxidants, phlogistins, metals, or inhibitors.

34. A method of preventing ocular oxidative stress and inflammation in mammals comprising the steps of:
assessing a value of at least one indicator factor in said mammals wherein said at least one indicator factor is correlated to a treatment of ocular oxidative stress and inflammation;
comparing said value of the at least one indicator factor to baseline values in other mammals with ocular oxidative stress and inflammation; and,
treating said mammals for ocular oxidative stress and inflammation based on a result of the previous step.

35. A method of preventing ocular oxidative stress and inflammation in mammals according to claim 34 wherein said at least one indicator factor is a level of reduced glutathione (GSH) in said mammal's blood.

36. A method of preventing ocular oxidative stress and inflammation in mammals according to claim 34 wherein said at least one indicator factor is a level of nuclear factor kappa-b (NFκB) activity in said mammal.

37. A method of preventing ocular oxidative stress and inflammation in mammals according to claim 34 wherein said at least one indicator factor is a level of oxidized glutathione (GSSG) in said mammal's blood.

38. A method of preventing ocular oxidative stress and inflammation in mammals according to claim 34 wherein said at least one indicator factor is a level of nitric oxide (NO) in said mammal's blood.

39. A method of preventing ocular oxidative stress and inflammation in mammals according to claim 34 wherein said at least one indicator factor is a level of vascular endothelial growth factor (VEGF) in said mammal's blood.

40. A method of preventing ocular oxidative stress and inflammation in mammals as described in claim 34 wherein the at least one indicator factor is a ratio of a level of oxidized glutathione (GSSG) to a level of reduced glutathione (GSH) in said mammal's blood.

41. A method of preventing ocular oxidative stress and inflammation in mammals according to claim 34 wherein said at least one indicator factor is selected from a group consisting of: cytokines, genes, proteins, lipids, minerals, growth factors, proteases, oxidants, antioxidants, phlogistins, metals, or inhibitors.

42. A method of preventing ocular oxidative stress and inflammation in mammals according to claim 34 wherein said treatment of the stress and inflammation comprises administering N-acetylcysteine to said mammal.

43. A method of assessing the progression of ocular oxidative stress and inflammation in mammals with ocular oxidative stress and inflammation comprising the steps of:
assessing a first value of at least one indicator factor in said mammals wherein said at least one indicator factor is correlated to ocular oxidative stress and inflammation;
assessing a second value of the at least one indicator; and,
comparing said second value to said first value of the at least one indicator factor.

44. A method of assessing the progression of ocular oxidative stress and inflammation in mammals according to claim 43 wherein said at least one indicator factor is a level of reduced glutathione (GSH) in said mammal's blood.

45. A method of assessing the progression of ocular oxidative stress and inflammation in mammals according to claim 43 wherein said at least one indicator factor is a level of oxidized glutathione (GSSG) in said mammal's blood.

46. A method of assessing the progression of ocular oxidative stress and inflammation in mammals according to claim 43 wherein said at least one indicator factor is a level of nuclear factor kappa-b (NFκB) activity in said mammal.

47. A method of assessing the progression of ocular oxidative stress and inflammation in mammals according to claim 43 wherein said at least one indicator factor is a level of vascular endothelial growth factor (VEGF) in said mammal's blood.

48. A method of assessing the progression of ocular oxidative stress and inflammation in mammals according to claim 43 wherein said at least one indicator factor is a level of nitric oxide (NO) in said mammal's blood.

49. A method for assessing the progression of ocular oxidative stress and inflammation in mammals as described in claim 43 wherein the at least one indicator factor is a ratio of a level of oxidized glutathione (GSSG) to a level of reduced glutathione (GSH) in said mammal's blood.

50. A method of assessing the progression of ocular oxidative stress and inflammation in mammals according to claim 43 wherein said at least one indicator factor is selected from a group consisting of: cytokines, genes, proteins, lipids, minerals, growth factors, proteases, oxidants, antioxidants, phlogistins, metals, or inhibitors.

51. A method of measuring ocular disease in mammals comprising the steps of:
assessing a first value of at least one indicator factor in said mammals wherein said at least one indicator factor is correlated to ocular oxidative stress and inflammation;
assessing a second value of the at least one indicator; and,
comparing said second value to said first value of the at least one indicator factor.

52. A method of assessing the treatment of ocular disease in mammals as described in claim 51 wherein said ocular diseases are selected from the group consisting of:
age-related macular degeneration, diabetic retinopathy, retinopathy, iritis, uveitis, keratitis, vasculitis, conjunctivitis, cataract, congenital ocular abnormalities, acquired ocular abnormalities, or glaucoma..

53. A method of measuring ocular disease as described in claim 51 wherein said at least one indicator factor is a level of reduced glutathione (GSH) in said mammal

54. A method of measuring ocular disease as described in claim 51 wherein said at least one indicator factor is a level of nuclear factor kappa-b (NFκK) activity in said mammal.

55. A method of measuring ocular disease as described in claim 51 wherein said at least one indicator factor is a level of nitric oxide (NO) in said mammal's blood.

56. A method of measuring ocular disease as described in claim 51 wherein said at least one indicator factor is a level of vascular endothelial growth factor (VEGF) in said mammal's blood.

57. A method of measuring ocular disease as described in claim 51 wherein said at least one indicator factor is a level of oxidized glutathione (GSSG) in said mammal's blood.

58. A method for measuring ocular disease as described in claim 51 wherein the at least one indicator factor is a ratio of a level of oxidized glutathione (GSSG) to a level of reduced glutathione (GSH) in said mammal's blood.

59. A method of measuring ocular disease as described in claim 51 wherein said at least one indicator factor is selected from a group consisting of: cytokines, genes, proteins, lipids, minerals, growth factors, proteases, oxidants, antioxidants, phlogistins, metals, or inhibitors.

60. A method of predicting ocular disease in mammals comprising the steps of:
assessing a value of at least one indicator factor in said mammals wherein said at least one indicator factor is correlated to a treatment of ocular oxidative stress and inflammation; and,
comparing said value of the at least one indicator factor to baseline values in other mammals in various stages of ocular oxidative stress and inflammation.

61. A method of predicting ocular disease in mammals as described in claim 60 wherein said ocular diseases are selected from the group consisting of: age-related macular degeneration, diabetic retinopathy, iritis, uretis, conjunctivitis, cataract or glaucoma.

62. A method of predicting ocular disease in mammals as described in claim 60 wherein said at least one indicator factor is a level of reduced glutathione (GSH) in said mammal's blood.

63. A method of predicting ocular disease in mammals as described in claim 60 wherein said at least one indicator factor is a level of nuclear factor kappa-b (NFκB) activity in said mammal.

64. A method of predicting ocular disease in mammals as described in claim 60 wherein said at least one indicator factor is a level of oxidized glutathione (GSSG) in said mammal's blood.

65. A method of predicting ocular disease in mammals as described in claim 60 wherein said at least one indicator factor is a level of nitric oxide (NO) in said mammal's blood.

66. A method of predicting ocular disease in mammals as described in claim 60 wherein said at least one indicator factor is a level of vascular endothelial growth factor (VEGF) in said mammal's blood.

67. A method of predicting ocular disease in mammals as described in claim 60 wherein the at least one indicator factor is a ratio of a level of oxidized glutathione (GSSG) to a level of reduced glutathione (GSH) in said mammal's blood.

68. A method of predicting ocular disease in mammals as described in claim 60 wherein said at least one indicator factor is selected from a group consisting of: cytokines, genes, proteins, lipids, minerals, growth factors, proteases, oxidants, antioxidants, phlogistins, metals, or inhibitors.

69. A method of assessing the treatment of ocular disease in mammals comprising the steps of:
assessing a first value of at least one indicator factor in said mammals wherein said at least one indicator factor is correlated to a treatment of ocular oxidative stress and inflammation;
treating said oxidative stress and inflammation;
assessing a second value of said at least one indicator factor; and,
comparing said second value to said first value of the at least one indicator factor.

70. A method of assessing the treatment of ocular disease in mammals as described in claim 69 wherein said ocular diseases are selected from the group consisting of:
age-related macular degeneration, diabetic retinopathy, retinopathy, iritis, uveitis, keratitis, vasculitis, conjunctivitis, cataract, congenital ocular abnormalities, acquired ocular abnormalities, or glaucoma.

71. A method of assessing the treatment of ocular disease in mammals as described in claim 69 wherein the at least one indicator factor is a level of reduced glutathione (GSH) in said mammal's blood.

72. A method of assessing the treatment of ocular disease in mammals as described in claim 69 wherein the at least one indicator factor is a level of oxidized glutathione (GSSG) in said mammal's blood.

73. A method of assessing the treatment of ocular disease in mammals as described in claim 69 wherein the at least one indicator factor is a level of nuclear factor kappa-b (NFκB) activity in said mammal.

74. A method of assessing the treatment of ocular disease in mammals as described in claim 69 wherein the at least one indicator factor is a level of vascular endothelial growth factor (VEGF) in said mammal's blood.

75. A method of assessing the treatment of ocular disease in mammals as described in claim 69 wherein the at least one indicator factor is a level of nitric oxide (NO) in said mammal's blood.

76. A method of assessing the treatment of ocular disease in mammals as described in claim 69 wherein the at least one indicator factor is a ratio of a level of oxidized glutathione (GSSG) to a level of reduced glutathione (GSH) in said mammal's blood.

77. A method of assessing the treatment of ocular disease in mammals as described in claim 69 wherein the at least one indicator factor is selected from a group consisting of: cytokines, genes, proteins, lipids, minerals, growth factors, proteases, oxidants, antioxidants, phlogistins, metals, or inhibitors.

78. A method of assessing the treatment of ocular disease in mammals as described in claim 69 wherein the treatment of said ocular disease comprises administering N-acetylcysteine to said mammal.

79. A method of assessing the progression of ocular disease in mammals comprising the steps of:
assessing a first value of at least one indicator factor in said mammals wherein said at least one indicator factor is correlated to ocular disease;
assessing a second value of the at least one indicator; and,
comparing said second value to said first value of the at least one indicator factor.

80. A method of assessing the treatment of ocular disease in mammals as described in claim 79 wherein said ocular diseases are selected from the group consisting of:
age-related macular degeneration, diabetic retinopathy, retinopathy, iritis, uveitis, keratitis, vasculitis, conjunctivitis, cataract, congenital ocular abnormalities, acquired ocular abnormalities, or glaucoma.

81. A method of assessing the progression of ocular disease mammals as described in claim 79 wherein said at least one indicator factor is a level of reduced glutathione (GSH) in said mammal's blood.

82. A method of assessing the progression of ocular disease mammals as described in claim 79 wherein said at least one indicator factor is a level of oxidized glutathione (GSSG) in said mammal's blood.

83. A method of assessing the progression of ocular disease mammals as described in claim 79 wherein said at least one indicator factor is a level of nuclear factor kappa-b (NFκB) activity in said mammal.

84. A method of assessing the progression of ocular disease mammals as described in claim 79 wherein said at least one indicator factor is a level of nitric oxide (NO) in said mammal's blood.

85. A method of assessing the progression of ocular disease mammals as described in claim 79 wherein said at least one indicator factor is a level of vascular endothelial growth factor (VEGF) in said mammal's blood.

86. A method of assessing the progression of ocular disease in mammals as described in claim 79 wherein the at least one indicator factor is a ratio of a level of oxidized glutathione (GSSG) to a level of reduced glutathione (GS H) in said mammal's blood.

87. A method of assessing the progression of ocular disease mammals as described in claim 79 wherein said at least one indicator factor is s elected from a group consisting of: cytokines, genes, proteins, lipids, minerals, growth factors, proteases, oxidants, antioxidants, phlogistins, metals, or inhibitors.

88. A method of diagnosing ocular disease in mammals comprising the steps of:
assessing a value of at least one indicator factor in said mammals wherein said at least one indicator factor is correlated to a treatment of ocular oxidative stress and inflammation; and,
comparing said value of the at least one indicator factor to baseline values in other mammals with ocular oxidative stress and inflammation.

89. A method of assessing the treatment of ocular disease in mammals as described in claim 88 wherein said ocular diseases are selected from the group consisting of:
age-related macular degeneration, diabetic retinopathy, retinopathy, iritis, uveitis, keratitis, vasculitis, conjunctivitis, cataract, congenital ocular abnormalities, acquired ocular abnormalities, or glaucoma.

90. A method of diagnosing ocular disease in mammals as described in claim 88 wherein said at least one indicator factor is a level of reduced glutathione (GSH) in said mammal's blood.

91. A method of diagnosing ocular disease in mammals as described in claim 88 wherein said at least one indicator factor is a level of oxidized glutathione (GSSG) in said mammal's blood.

92. A method of diagnosing ocular disease in mammals as described in claim 88 wherein said at least one indicator factor is a level of nuclear factor kappa-b (NFκB) activity in said mammal.

93. A method of diagnosing ocular disease in mammals as described in claim 88 wherein said at least one indicator factor is a level of vascular endothelial growth factor (VEGF) in said mammal's blood.

94. A method of diagnosing ocular disease in mammals as described in claim 88 wherein said at least one indicator factor is a level of nitric oxide (NO) in said mammal's blood.

95. A method of diagnosing ocular disease in mammals as described in claim 88 wherein the at least one indicator factor is a ratio of a level of oxidized glutathione (GSSG) to a level of reduced glutathione (GSH) in said mammal's blood.

96. A method of diagnosing ocular disease in mammals as described in claim 88 wherein said at least one indicator factor is selected from a group consisting of:
cytokines, genes, proteins, lipids, minerals, growth factors, proteases, oxidants, antioxidants, phlogistins, metals, or inhibitors.

97. A method of preventing ocular diseases in mammals comprising the steps of:
assessing a value of at least one indicator factor in said mammals wherein said at least one indicator factor is correlated to a treatment of ocular oxidative stress and inflammation;
comparing said value of the at least one indicator factor to baseline values in other mammals with ocular oxidative stress and inflammation; and,
treating said mammals for ocular oxidative stress and inflammation based on a result of the previous step.

98. A method of assessing the treatment of ocular disease in mammals as described in claim 97 wherein said ocular diseases are selected from the group consisting of:
age-related macular degeneration, diabetic retinopathy, retinopathy, iritis, uveitis, keratitis, vasculitis, conjunctivitis, cataract, congenital ocular abnormalities, acquired ocular abnormalities, or glaucoma.

99. A method of preventing ocular disease in mammals as described in claim 97 wherein the at least one indicator factor is a level of reduced glutathione (GSH) in said mammal's blood.

100. A method of preventing ocular disease in mammals as described in claim 97 wherein the at least one indicator factor is a level of oxidized glutathione (GSSG) in said mammal's blood.

101. A method of preventing ocular disease in mammals as described in claim 97 wherein the at least one indicator factor is a level of nuclear factor kappa-b (NFκB) activity in said mammal.

102. A method of preventing ocular disease in mammals as described in claim 97 wherein the at least one indicator factor is a level of nitric oxide (NO) in said mammal's blood.

103. A method of preventing ocular disease in mammals as described in claim 97 wherein the at least one indicator factor is a level of vascular endothelial growth factor (VEGF) in said mammal's blood.

104. A method of preventing ocular disease in mammals as described in claim 97 wherein the at least one indicator factor is a ratio of a level of oxidized glutathione (GSSG) to a level of reduced glutathione (GSH) in said mammal's blood.

105. A method of preventing ocular disease in mammals as described in claim 97 wherein the at least one indicator factor is s elected from a group consisting of: cytokines, genes, proteins, lipids, minerals, growth factors, proteases, oxidants, antioxidants, phlogistins, metals, or inhibitors.

106. A method of preventing ocular disease in mammals as described in claim 97 wherein said treatment of said ocular disease comprises administering N-acetylcysteine to said mammal.
